(19) United States
(12) Patent Application Publication
(10) Pub. No.: US 2017/0028005 A1
(43) Pub. Date: Feb. 2, 2017

(54) NATURAL SWEETENER

(71) Applicant: NATGOOD IP, LLC, Houston, TX (US)

(72) Inventor: Ryan Turner, Houston, TX (US)

(73) Assignee: NATGOOD IP, LLC, Houston, TX (US)

(21) Appl. No.: 15/291,710

(22) Filed: Oct. 12, 2016

Related U.S. Application Data
(63) Continuation-in-part of application No. 15/158,709, filed on May 19, 2016.
Application No. 15/158,709 is a continuation-in-part of application No. 14/610,647, filed on Jan. 30, 2015.
(60) Provisional application No. 62/048,617, filed on Sep. 10, 2014.
(60) Provisional application No. 61/933,673, filed on Jan. 30, 2014.

(51) Int. Cl. A61K 36/42; A61K 31/19; A23L 33/105; A61K 9/00; A23L 29/30; A61K 31/047; A61K 31/7004

(52) U.S. Cl. CPC A61K 36/42; A61K 31/047; A61K 31/19; A61K 31/7004; A61K 9/0053; A23L 29/37; A23L 33/105; A23V 2002/00

(57) ABSTRACT

A method of attenuating a postprandial insulin response comprising administering to a subject a composition comprising at last one sugar alcohols, monk fruit, a rheology modifier, and tagatose wherein a subject ingesting a carbohydrate-containing food comprising the composition experiences a decrease in the postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

### CROSS-REFERENCE TO RELATED APPLICATIONS

The present application is a continuation-in-part of and claims priority to U.S. patent application Ser. No. 15/158,709 filed May 19, 2016 and titled “Natural Sweetener,” which is a continuation-in-part of U.S. patent application Ser. No. 14/610,647 filed Jan. 30, 2015 and titled “Natural Sweetener,” which claims the benefit of Provisional Application Ser. No. 62/048,617 filed Sep. 10, 2014 and also claims the benefit of Provisional Application Ser. No. 61/933,673 filed Jan. 30, 2014. All the noted applications are incorporated by reference herein as if reproduced in full below.

### TECHNICAL FIELD

This disclosure relates to sweetener compositions. More specifically this disclosure relates to compositions for use as natural sweeteners and methods of ameliorating adverse metabolic conditions.

### BACKGROUND

In 2000, according to the USDA, each individual in the U.S. consumed over 150 pounds of caloric sweeteners as part of the standard American diet (SAD). The SAD typically includes highly processed, calorie-dense, nutrient-depleted foods that results in exaggerated postprandial spikes in blood glucose and lipids. This state, called postprandial dysmetabolism, induces oxidant stress, which increases in direct proportion to the increases in glucose and triglycerides after a meal. This transient increase in free radicals acutely triggers atherogenic changes including inflammation, endothelial dysfunction, hypercoagulability, and sympathetic hyperactivity. Postprandial dysmetabolism is an independent predictor of future cardiovascular events even in nondiabetic individuals.

For diabetic individuals who are at increased risk of developing microvascular complications (retinopathy, nephropathy, and neuropathy) and cardiovascular disease (CVD) the Diabetes Control and Complications Trial (DCCT) and U.K. Prospective Diabetes Study (UKPDS) showed that treatment programs resulting in improved glycemic control reduced the microvascular complications of diabetes. Unfortunately, the majority of patients with diabetes fail to achieve their glycemic goals. Elevated postprandial glucose concentrations may contribute to suboptimal glycemic control. Postprandial hyperglycemia is also one of the earliest abnormalities of glucose homeostasis associated with type 2 diabetes and is markedly exaggerated in diabetic patients with fasting hyperglycemia.

Reduced calorie or non-caloric natural sweeteners are touted as offering a healthful alternative to artificial sweeteners but current products have a variety of challenges associated with their use including caloric content, taste, and appearance. Thus, an ongoing need exists for natural sweeteners having desirable characteristics.

### SUMMARY

Disclosed herein is a method of attenuating a postprandial insulin response comprising administering to a subject a composition comprising at last one sugar alcohol, stevia, monk fruit, a rheology modifier, and tagatose wherein a subject ingesting a carbohydrate-containing food comprising the composition experiences a decrease in the postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

Also disclosed herein is a method of attenuating a postprandial glucose response comprising administering to a subject a composition comprising at last one sugar alcohol, stevia, monk fruit, a rheology modifier, and tagatose wherein a subject ingesting (i) a carbohydrate-containing food and (ii) the composition experiences a decrease in a postprandial insulin response of from about 10% to about 90% for a time period ranging from about 15 minutes to about 180 minutes subsequent to ingestion of the composition when compared to the postprandial insulin response of a subject ingesting an otherwise identical carbohydrate-containing food comprising an equivalent amount of sucrose.

### BRIEF DESCRIPTION OF THE DRAWINGS

FIG. 1 depicts plots of the time-intensity curves of common sweeteners and sucrose.

FIG. 2 depicts plots of the change in blood glucose level as a function of time for both a sweetener composition and sugar.

FIG. 3 is a plot of the change in blood glucose level as a function of time following ingestion of the sweetener compositions of example 2.

FIGS. 4 and 5 are overlays of the UV spectrum for the unagglomerated NSC, the agglomerated NSC, and acetonitrile.

FIGS. 6-8 are electrospray ionization mass spectra for the samples of Example 4.

FIGS. 9A and 9B are a comparison of the electrospray ionization mass spectra for the samples of Example 4.

FIG. 10 is an aspect of an electrospray ionization mass spectra of a repressive sweetener composition of the type disclosed herein.

FIG. 11 is a plot of the postprandial glucose response as a function of time subsequent to ingestion of either an NSC of the type disclosed herein or sucrose.

FIG. 12 is a plot of the postprandial insulin response as a function of time subsequent to ingestion of either an NSC of the type disclosed herein or sucrose.

FIG. 13 is a plot of the postprandial fructose response as a function of time subsequent to ingestion of either an NSC of the type disclosed herein or sucrose.

### DETAILED DESCRIPTION

Disclosed herein are natural sweetener compositions (NSCs). Also disclosed herein are ingestible compositions (both edible and nonedible) comprising the NSC. In an aspect, the NSC comprises at least one stevia extract, at least one monk fruit extract, at least one sugar alcohol, and tagatose. An NSC of the type disclosed herein may optionally further comprise a blend of one or more natural flavors (i.e., a flavor blend). In some aspects, the NSC further comprises a rheology modifier.

In an aspect, the compositions disclosed herein are characterized by a low glycemic index when compared to table sugar (i.e., sucrose). The NSCs disclosed herein may also be characterized by the ability to attenuate the blood glucose response to consumption of other carbohydrate-containing foods and as such are characterized as repressive sweetener compositions. The NSCs disclosed herein may also mitigate a subject's response to glucose and/or fructose and result in an attenuated insulin response. Also disclosed herein are methods of ameliorating postprandial dysmetabolism and related dysfunctions comprising administering to a subject an NSC of the type disclosed herein.

In an aspect, the NSC comprises at least one extract of stevia. Herein, the term extract refers to a preparation containing the active ingredient of a substance in a concentrated form. Stevia extract is known to be a source of steviol glycosides which are responsible for the sweet taste of the leaves of the stevia plant (Stevia rebaudiana). Steviol glycosides range in sweetness from 40 to 300 times sweeter than sucrose and are also characterized as being heat-stable, pH-stable, and non-fermentable. In an aspect, the NSC comprises stevia in an amount of from about 0.01 weight percent (wt.)% to about 3 wt. %, alternatively from about 0.02 wt. % to 1.8 wt. %, or alternatively from about 0.05 wt. % to about 0.8 wt. % based on the total weight of the NSC. The stevia may be a component of an extract where the stevia is present in densities ranging from 0.1 g/ml to 20 g/ml, alternatively about 0.1 g/ml to 20 g/ml or alternatively from about 0.5 g/ml to about 20 g/ml.

In an aspect, the NSC comprises monk fruit extract. Monk fruit is obtained from the plant Siraitia grosvenorii which is a herbaceous perennial vine of the Cucurbitaceae (gourd) family, native to southern China and northern Thailand. In an aspect, the NSC comprises monk fruit in an amount of from about 0.05 wt. % to about 20 wt. %, alternatively from about 0.09 wt. % to about 20 wt. %, or alternatively from about 0.1 wt. % to about 20 wt. % based on the total weight of the NSC. The monk fruit may be a component of an extract where the monk fruit is present in densities ranging from 0.1 g/ml to 20 g/ml, alternatively about 1 g/ml to 5 g/ml or alternatively about 1 g/ml.

In an aspect, the NSC comprises a sugar alcohol, alternatively at least two sugar alcohols, or alternatively at least three sugar alcohols. Sugar alcohols, also known as polyols, polyhydric alcohols, or polyalcohols, are the hydrogenated forms of the aldoses or ketoses of a sugar. For example, glucitol, also known as sorbitol, has the same linear structure as the chain form of glucose, but the aldehyde (—CHO) group is replaced with a —CH₂OH group. Other common sugar alcohols include the monosaccharides erythritol and xylitol and the disaccharide maltitol. In an aspect, the sugar alcohol comprises erythritol, mannitol, sorbitol, arabitol, threitol, xylitotl, ribitol, galactitol, fruitol, iditol, inositol, volemitol, lactitol, malitol, or combinations thereof. In an aspect, the sugar alcohol comprises maltitol. In an aspect, the sugar alcohols are present in the NSC in a combined amount of from about 20 wt. % to about 80 wt. %, alternatively from about 30 wt. % to about 70 wt. %, or alternatively from about 38 wt. % to about 60 wt. % based on the total weight of the NSC.

In an aspect, the NSC comprises tagatose. Tagatose, or (3S,4S,5R)-1,3,4,5,6-pentahydroxy-hexan-2-one, is a naturally-occurring monosaccharide with the chemical formula C₆H₁₂O₆ (depicted in Structure I).

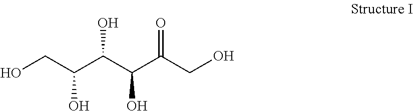 [SMILES: O=C(CO)[C@@H](O)[C@@H](O)[C@H](O)CO]

Tagatose has been a GRAS consumable since 2001. In an aspect, tagatose is present in the NSC in an amount of from about 20 wt. % to about 95 wt. %, alternatively from about 30 wt. % to about 70 wt. %, or alternatively from about 40 wt. % to about 60 wt. % based on the total weight of the NSC.

The NSC may optionally comprise a blend of at least two flavors (i.e., a flavor blend) to create an enhancing natural flavor profile. Nonlimiting examples of natural flavors suitable for use in the flavor blend include oils or extracts having flavors such as thaumatin; citrus fruits; berries; orange; lemon; lime; tangerine; mandarin; grapefruit; acerola; grape; pear; passion fruit; pineapple; banana; apple; cranberry; cherry; raspberry; chokeberry; peach; plum; currant; black currant; cranberry; blackberry; pomegranate; acai; noni; elderberry; goji berry; rosehips; bilberry; hawthome berry; ginko; goru kola; rooibos; boysenberry; catuaba; horny goat weed; yohimbe; damiana; red raspberry leaf; vitex berry; blessed thistle; wolfberry; strawberry; mirabelle; watermelon; honeydew; cantaloupe; mango; papaya; botanical flavors derived from cola; tea; coffee; vanilla; almond; vegetables; tomato; cabbage; celery; cucumber; spinach; carrot; lettuce; watercress; dandelion; rhubarb; beet; cocona; cocoa; guava; Japanese knotweed; han guo, green tea, white tea, and any available tea blends; grapeseed; blueberries; or combinations thereof. In an aspect, the flavor blend is included in the NSC any amount suitable to meet some user and/or process need. For example, the amount of flavor blend included may be designed to provide a desired organoleptic effect. In an aspect, the flavor blend is present in the NSC an amount of from about 0.001 wt. % to about 0.095 wt. %, alternatively from about 0.002 wt. % to about 0.050 wt. %, or alternatively from about 0.005 wt. % to about 0.010 wt. % based on the total weight of the NSC.

In an aspect, the NSC further comprises a rheology modifier. A rheology modifier suitable for inclusion in the NSC may comprise any material compatible with the other components of the NSC and able to provide the compositional properties disclosed herein. In an aspect, the rheology modifier comprises a polysaccharide, alternatively a polysaccharide gum. A polysaccharide gum suitable for use in the present disclosure may be further characterized as being hydrocolloidal. Herein the term “hydrocollodial” takes on its standard meaning of a substance that forms a gel in the presence of water.

A polysaccharide gum suitable for use in the NSC may be derived from natural sources or may be prepared synthetically. In an aspect, the polysaccharide gum comprises alginate, carrageenan, xanthan, pectin, gellan, welan, pullulan, curdlan, rhamsan, sphingan polymers, or combinations thereof. In an aspect, the polysaccharide gum may be plant-derived. For example, carrageenan can extracted from certain species of the class Rhodophyceae (red seaweed) while xanthan gum, for example, is a polysaccharide that can be produced by fermentation using Xanthomonas bacterium. Gellan gum is the generic name of a polysaccharide that can be produced by cultured Pseudomonas elodea or related organisms. Curdlan is a polysaccharide gum that can be produced by a microorganism (e.g., Alcaligenes faecalis varmyxogenes). Alginate is a polysaccharide gum that can be obtained by extraction from certain species of seaweed, or alternatively Azotobacter vinelandii or Pseudomonas aeruginosa can be used to produce the polysaccharide gum through fermentation. In an aspect, the polysaccharide gum comprises xanthan gum.

The backbone of the xanthan gum structure consists of two β-D-glucose units linked through the 1 and 4 positions. The side chain consists of two mannose and one glucuronic acid, so the chain consists of repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. In an aspect, xanthan gum suitable for use in the present disclosure exhibits pseudoplastic behavior which is observable a wide range of temperatures (e.g., 90° C. to 260° C.). Herein pseudoplasticity is a term that refers to the reduction of viscosity when shear forces are applied.

In an aspect, the rheology modifier is present in the NSC in an amount ranging from about 0.01 wt. % to about 10 wt. %, alternatively from about 0.1 wt. % to about 10 wt. %, or alternatively from about 0.5 wt. % to about 9 wt. % based on the total weight of the NSC.

In an aspect, the components of an NSC of the type disclosed herein may be combined in any order and the composition prepared using any suitable methodology. Alternatively, an NSC of the type disclosed herein is formed into a particulate/powder material using techniques such as fluid bed agglomeration. Herein, fluid bed agglomeration refers to a process involving suspension of particles in an air stream followed by the spraying of a liquid onto the fluidized bed (top-down spray). Particles in the path of the spray develop an adhesive quality (i.e., become sticky) and upon collision with other particles in the bed adhere and form granules. The process may be continued until the granules develop some user and/or process desired characteristics.

In an aspect, the NSC may be prepared by the mixing of all components of the NSC with the exception of the flavor blend in the fluidized bed. Agglomeration of the NSC may be accomplished by the spraying of the flavor blend onto the fluidized particles. For example, an NSC may be prepared by (i) dry blending in a fluidized bed a rheology modifier, tagatose, stevia extract, xanthan gum, a flavor blend, erythritol, and a second sugar alcohol to form a first mixture; (ii) spray depositing an aqueous solution (e.g., aqueous flavor blend) onto the first mixture to generate a wet mixture; and (iii) removing excess moisture from the wet mixture to generate granules.

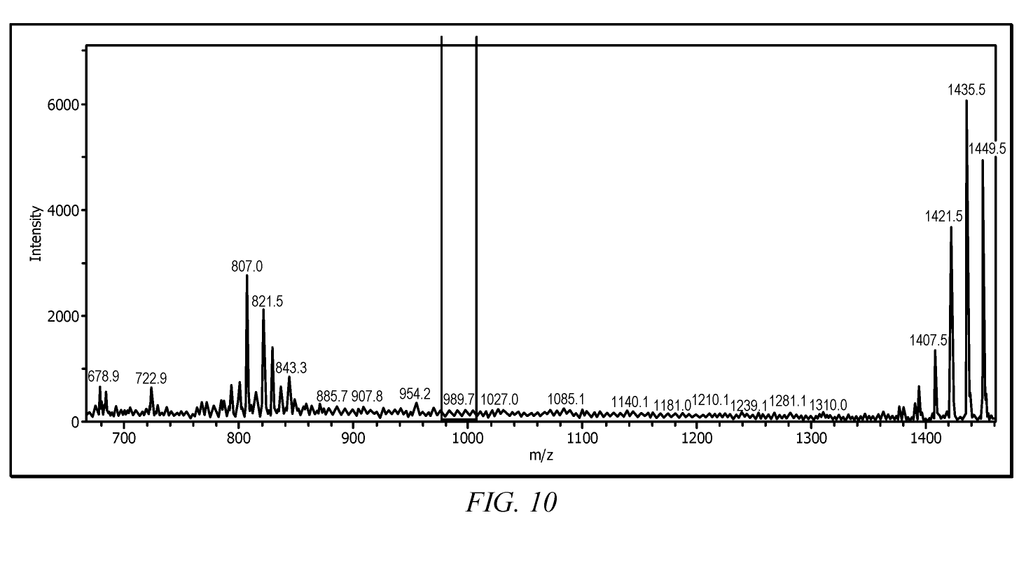

Further, an NSC of the type disclosed herein may form agglomerated particles having a characteristic fragmentation pattern when subjected to electrospray ionization (ESI) mass spectrometry. In an aspect, an NSC when subjected ESI mass spectrometry is characterized by a mass spectrum with a mass to charge ratio (m/z) in the range of about 960 to 990 that has a signal intensity reduced by greater than about 90% when compared to the mass spectrum of an unagglomerated NSC in the same m/z range. Alternatively, an agglomerated NSC of the type disclosed herein is characterized by an electrospray ionization mass spectrum with a mass to charge ratio (m/z) in the range of about 960 to 990 that has a signal intensity reduced by greater than about 95% when compared to the mass spectrum of an unagglomerated NSC in the same m/z range. Alternatively, an NSC of the type disclosed herein has a signal intensity in the m/z range of about 960 to 990 that is ±10% of the baseline spectrum alternatively ±7%, or alternatively ±5%. In an aspect, an NSC of the type disclosed herein has the electrospray mass ionization spectrum of FIG. 10.

In an aspect, the NSC may be subjected to fluid bed agglomeration until a particular particle size distribution of the composition is reached. For example, fluid bed agglomeration of the NSC may be carried out to provide a mean particle size distribution ranging from about 250 microns to about 700 microns, alternatively from about 275 microns to about 500 microns, or alternatively from about 300 microns to about 400 microns. In an aspect, the NSC may comprise a maximum of about 25% of particles having a mean particle size of less than about 200 microns. Further, the NSC may comprise a maximum of about 25% of particles having a mean particle size of greater than about 800 microns. NSCs of the type disclosed herein are characterized by a multimodal particle size distribution having a uniformity within each modality of the multimodal particle size distribution of ±20%, alternatively ±15%, or alternatively ±10%. Herein, a “multimodal” particle size distribution refers to the NSC having a plot of the cumulative volume (or indicator thereof) versus particle size characterized by the presence of more than one maxima or “mode” corresponding to different particle size ranges. In an aspect, the NSC has a bimodal particle size distribution.

Particle size distribution of the NSC may impact manufacturing of the materials from the production and processing side to the appearance of the product to the user. An NSC having the disclosed particle size features may be characterized by the rapid dissolution of particles in hot and cold systems, reduced separation of the NSC particle when it is a component of a dry mix, reduced dust generation, and the presence of larger particle sizes that provides a positive visual impact.

In an aspect, the NSC has an apparent density, also known as a bulk density, comparable to that of table sugar. Herein, the apparent density refers to the mass of a unit volume of the NSC, which can be freely (without pressing or tapping) poured into a container. An NSC of the type disclosed herein may be characterized by an apparent density that is within about ±30% of the apparent density of table sugar, alternatively about ±20% of the apparent density of table sugar, or alternatively about ±10% of the apparent density of table sugar. For example, the apparent density of the NSC may be from about 750 kg/m³ to about 940 kg/m³, alternatively from about 775 kg/m³ to about 925 kg/m³, or alternatively from about 800 kg/m³ to about 875 kg/m³ and be determined in accordance with ISO 697.

In an aspect, the NSC has a compressibility that is increased when compared to that of table sugar. Herein, the compressibility refers to the degree to which the NSC powder compacts as a function of applied pressure. The compressibility of an NSC can be evaluated by calculating the compressibility index. The compressibility index is determined by measuring the unsettled apparent volume, Vₒ, and the final tapped volume of the material (i.e., NSC), V_{f}, after tapping the material until no further volume change occurs. The compressibility index is then calculated using the equation:

Compressibility index=100%×(Vₒ−V_{f}/Vₒ)

The NSC may be characterized by a compressibility that is about 10% greater than the compressibility of table sugar, alternatively that is about 20% greater than the compressibility of table sugar, or alternatively that is about 30% greater than the compressibility of table sugar. In an aspect, an NSC has a compressibility index of from about 1% to about 20%, alternatively from about 5% to about 20%, or alternatively from about 5% to about 10%.

In an aspect, the NSC has a flowability comparable to that of table sugar. Herein, the flowability refers to the ability of the NSC to flow through a calibrated orifice. For example, flowability is a consideration when using high-speed rotary tablet machines, in order to ensure homogenous and rapid flow of material for uniform die filling. During the short dwell-time (milliseconds) used in these high speed rotary tablet machines, the required amount of material should be transferred into the die cavities with a reproducibility of ±5%. The Hausner ratio is a metric for evaluating the flowability of a material and is calculated by dividing Vₒ by V_{f}. Hausner ratios less than 1.25 indicate a free flowing material. An NSC may be characterized by a flowability that is within about ±30% of the flowability of table sugar, alternatively about ±20% of the flowability of table sugar, or alternatively about ±10% of the flowability of table sugar. Further, the NSC may have a Hausner ratio of from about 0.1 to about 1.2, alternatively from about 0.5 to about 1.0, or alternatively from about 0.5 to about 0.75.

In an aspect, the NSC has a sugar recipe equivalence (SRE) of about 1. Herein, the SRE refers to the amount of NSC equivalent to the amount of table sugar in a particular recipe. For example, a SRE of about 1 indicates that for a recipe using 1 cup of table sugar, 1 cup of an NSC may be substituted. In an aspect, the NSC has an SRE of about 0.85, alternatively about 0.90, or alternatively 0.95.

In an aspect, the NSC has a moisture retention value comparable to that of table sugar. Herein, the moisture retention value refers to the extent to which the NSC binds water in an ingestible product. An NSC of the type disclosed herein may be characterized by a moisture retention value that is within about ±30% of the moisture retention value of table sugar, alternatively about ±20% of the moisture retention value of table sugar, or alternatively about ±10% of the moisture retention value of table sugar.

In an aspect, the NSC has a rise ratio of about 1. Herein, the rise ratio refers to the extent to which a baked good rises when prepared with table sugar in comparison to the rise of the same baked good in the presence of the NSC. In an aspect, the NSC has a rise ratio of about 0.85, alternatively about 0.90, or alternatively 0.95.

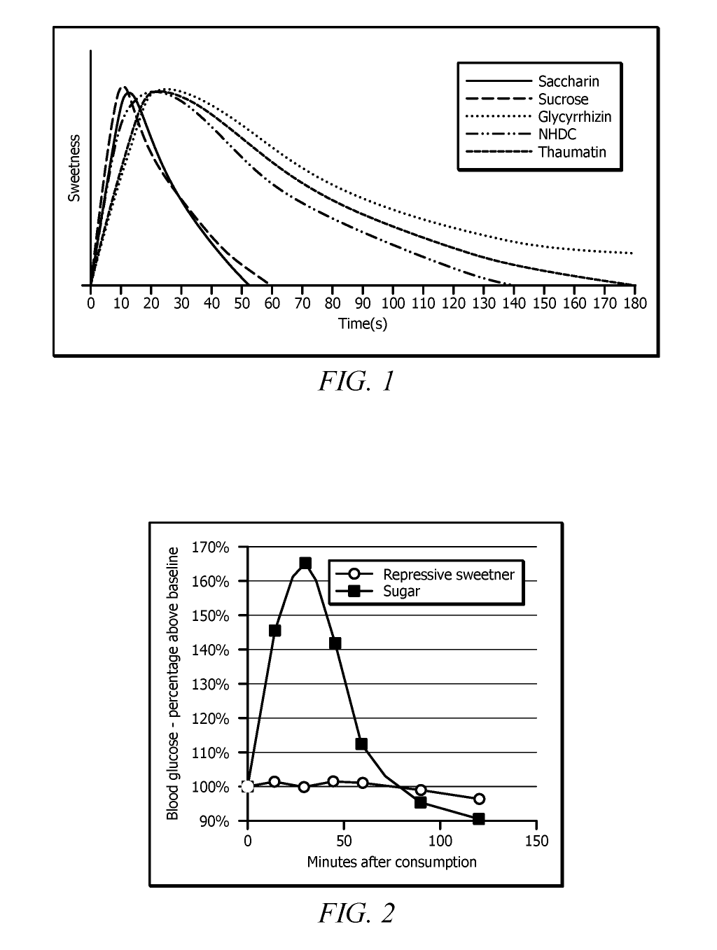

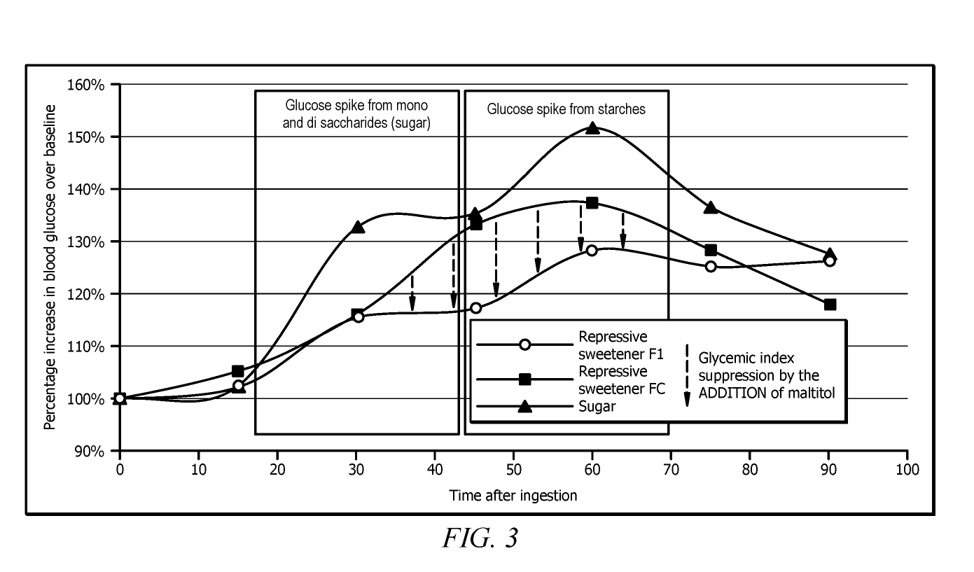

In an aspect, the NSC has a time-intensity curve that is similar to that of sucrose. Herein, the intensity refers to the intensity of sweetness. The time-intensity curves for common sweeteners and sucrose are presented in FIG. 1. The time intensity curve for the NSC may be sufficiently similar to that of sucrose so as allow formulations with citric acid that balance sweet and sour taste profiles. Sweetness intensity can be determined by descriptive testing which is a sensory analysis using human assessors (e.g., an expert panel) to evaluate a particular feature of an ingestible product. For example the flavor attributes of the sample (e.g., sweetness) are tracked via an appropriate computer program for a set time period following ingestion of a sample and the information used to prepare a time intensity profile.

In an aspect, an NSC of the type disclosed herein may be utilized as a sugar source in a fermentation process.

In an aspect, the NSC is characterized by a low glycemic index. Herein, the glycemic index (GI) refers to a number associated with carbohydrate-containing foods that indicates the food's effect on a person's blood glucose (also called blood sugar) level. A value of 100 represents the standard, an equivalent amount of pure glucose. The GI estimates how much each gram of available carbohydrate (total carbohydrate minus fiber) in a food raises a person's blood glucose level following consumption of the food, relative to consumption of pure glucose.

Glycemic index can be measured by any suitable methodology. For example, to determine a food's GI value, measured portions of the food containing 50 grams of available carbohydrate (or 25 grams of available carbohydrate for foods that contain lower amounts of carbohydrate) may be fed to 10 healthy people after an overnight fast. Finger-prick blood samples can be taken at 15-30 minute intervals over the next two hours. These blood samples can be used to construct a blood sugar response curve for the two hour period. The incremental area under the curve (iAUC) is calculated to reflect the total rise in blood glucose levels after eating the test food. The GI value is calculated by dividing the iAUC for the test food by the iAUC for the reference food (same amount of glucose) and multiplying by 100. The average of the GI ratings from all ten subjects is designated as the GI for that food. In an aspect, an NSC of the type disclosed herein has a GI value of from about 1 to about 10, alternatively from about 2 to about 7, or alternatively from about 2 to about 5 based on consumption of 50 g of available carbohydrates.

In an aspect, an NSC of the type disclosed herein is able to attenuate the blood glucose response of a subject that ingests carbohydrate-containing foods (CCF) subsequent to ingestion of the NSC. For example, from about 1 minute to about 3 hours subsequent to ingestion of an NSC of the type disclosed herein, ingestion of another CCF may produce a blood glucose response that is reduced by from about 5% to about 90% when compared to the blood glucose response produced without prior ingestion of the NSC. The repression of the blood glucose response subsequent to the ingestion of the NSC is predicated upon the ingestion of an effective amount of the NSC and may continue for a period of time ranging from about 5 minutes to about 2 hours or alternatively from about 15 minutes to about 1 hour following ingestion of the NSC. In some aspects, when compared to the blood glucose response of the CCF ingested without prior ingestion of an NSC of the type disclosed herein, the blood glucose response after ingestion of the NSC is reduced by from about 10% to about 80%, or alternatively from about 20% to about 50%. The NSCs disclosed herein act as repressive sweetener compositions which reduce the blood glucose level response to ingestion of a CCF to the ranges disclosed herein.

In an aspect, an NSC of the type disclosed herein is able to attenuate the blood fructose response of a subject that ingests carbohydrate-containing foods (CCF) subsequent to ingestion of the NSC. For example, from about 1 minute to about 3 hours subsequent to ingestion of an NSC of the type disclosed herein, ingestion of another CCF may produce a blood fructose response that is reduced by from about 5% to about 90% when compared to the blood fructose response produced without prior ingestion of the NSC. The repression of the blood fructose response subsequent to the ingestion of the NSC is predicated upon the ingestion of an effective amount of the NSC and may continue for a period of time ranging from about 5 minutes to about 2 hours or alternatively from about 15 minutes to about 1 hour following ingestion of the NSC. In some aspects, when compared to the blood fructose response of the CCF ingested without prior ingestion of an NSC of the type disclosed herein, the blood fructose response after ingestion of the NSC is reduced by from about 10% to about 80%, or alternatively from about 20% to about 50%. The NSCs disclosed herein act as repressive sweetener compositions which reduce the blood fructose level response to ingestion of a CCF to the ranges disclosed herein.

In an aspect, an NSC of the type disclosed herein is characterized by a GI for the composition as a whole that is less than the sum of the GI values of the constituents (taking into account their weight percentage in the composition). An example of a NSC of the type disclosed herein is a composition comprising maltitol, erythritol, tagatose, monkfruit, a rheology modifier, stevia, and a flavor blend, and is characterized by a GI of from about 2 to about 6.

In an aspect, an NSC of the type disclosed herein is included in an edible composition such as a food product. “Food” herein refers to any edible product intended for consumption by humans or animals, including solids, semi-solids, or liquids (e.g., beverages). The term “food” and the term “food and beverage” are herein used interchangeably. Examples of food and beverage products or formulations include, but are not limited to sweet coatings, frostings, or glazes for comestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, the Sauces, Dressings, Condiments category, the Baby Food category, and/or the Spreads category.

In general, the Soup category refers to canned/preserved, dehydrated, instant, chilled, ultra-high temperature (UHT) and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

“Dehydrated and Culinary Food Category” herein refers to: (i) Cooking aid products such as: powders, granules, pastes, concentrated liquid products, including concentrated bouillon, bouillon and bouillon-like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid, or frozen.

The Beverage category herein refers to beverages, beverage mixes and concentrates, including but not limited to, carbonated and non-carbonated beverages, alcoholic and non-alcoholic beverages, ready-to-drink beverages, liquid concentrate formulations for preparing beverages such as sodas, and dry powdered beverage precursor mixes. The Beverage category also includes alcoholic drinks, soft drinks, sports drinks, isotonic beverages, and hot drinks. The alcoholic drinks include, but are not limited to beer, cider/perry, flavored alcoholic beverages, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The hot drinks include, but are not limited to coffee, such as fresh, instant, and combined coffee; tea, such as black, green, white, oolong, and flavored tea; and other hot drinks including flavor-, malt- or plant-based powders, granules, blocks or tablets mixed with milk or water.

The Snack Food category generally refers to any food that can be a light informal meal including, but not limited to sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream, sugar and flavoring. Examples of ice cream include, but are not limited to: impulse ice cream; take-home ice cream; frozen yogurt and artisanal ice cream; soy, oat, bean (e.g., red bean and mung bean), and rice-based ice creams.

The Confectionary category generally refers to edible products that are sweet to the taste. Examples of confectionary include, but are not limited to candies, gelatins, chocolate confectionery, sugar confectionery, gum, and the likes and any combination products.

The Meal Replacement category generally refers to any food intended to replace the normal meals, particularly for people having health and/or fitness concerns. Examples of meal replacements include, but are not limited to slimming products and convalescence products.

The Ready Meal category generally refers to any food that can be served as a meal without extensive preparation or processing. The ready meal category includes products that have had recipe “skills” added to them by the manufacturer, resulting in a high degree of readiness, completion and convenience. Examples of ready meals include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles.

The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to honey, chocolate spreads, nut-based spreads, and yeast-based spreads.

The Dairy Product category generally refers to edible products produced from mammal's milk. Examples of dairy product include, but are not limited to drinking milk products, cheese, yogurt and sour milk drinks, and other dairy products.

Exemplary foods include confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, powder mints, boiled sweets, pastilles, chews, toffees, caramels and nougat, medicated confectionery, lollipops, liquorice, other sugar confectionery, gum, chewing gum, sugarized gum, sugar-free gum, functional gum, bubble gum, cough drops, herbs, seeds, spices, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, ready-to-eat cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, fruit juices, vegetable juices, cream, cheese, processed cheese, spreadable processed cheese, unspreadable processed cheese, unprocessed cheese, spreadable unprocessed cheese, hard cheese, packaged hard cheese, unpackaged hard cheese, yogurt, plain/natural yogurt, flavored yogurt, fruited yogurt, probiotic yogurt, drinking yogurt, regular drinking yogurt, probiotic drinking yogurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded cereals and snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, yeast-based spreads, toppings, and syrups.

In one aspect, foods include alcoholic beverages, baby food, baby formula, baked goods, breakfast cereals, cheese, chewing gum, coffee whiteners, condiments and relishes, confectionary and frostings, crackers, dairy products, egg products, fats and oils, fish products, frozen dairy, frozen dinners, fruit ices, gelatins and puddings, grain mixtures, granulated sugar, imitation dairy products, coffee, coffee products and coffee beverages, jams and jellies, meat products, milk products, non-alcoholic beverages, nut products, grains and grain products, poultry, processed fruits, processed vegetables, reconstituted vegetables, ready to eat meals, salad dressings, seasonings and flavors, snack foods, soft candy, soups, sugar substitutes, sweet sauce, sweetener blends, table top sweeteners, tea, tea products, and tea beverages.

In an aspect, foods include table top sweeteners and beverages. Beverages include, but are not limited to, fruit juices, soft drinks, tea, coffee, beverage mixes, milk drinks, alcoholic and nonalcoholic beverages.

In an aspect, an NSC is included in a non-edible ingestible product. Herein, “non-edible ingestible products” refer to supplements, nutraceuticals, functional food products (e.g., any fresh or processed food claimed to have a health-promoting and/or disease-preventing properties beyond the basic nutritional function of supplying nutrients), pharmaceutical and over the counter products, oral care products such as dentifrices and mouthwashes, cosmetic products such as sweetened lip balms and other personal care products that use sucralose and/or other sweeteners. In general, over the counter (OTC) product and oral care product generally refer to product for household and/or personal use which may be sold without a prescription and/or without a visit to a medical professional. Examples of the OTC products include, but are not limited to vitamins and dietary supplements; topical analgesics and/or anesthetic; cough, cold and allergy remedies; antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g. muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care products include, but are not limited to mouth cleaning strips, toothpaste, toothbrushes, mouthwashes/dental rinses, denture care, mouth fresheners, at-home teeth whiteners and dental floss.

In an aspect, a food product comprises the NSC. In such aspects, the food product may be utilized after introduction of the NSC to the food product with no additional heat-treatment of the food product. In another aspect, the food product may be heat-treated (e.g., baked) subsequent to the addition of the NSC.

An NSC of the type disclosed herein may exclude one or more components that could adversely affect the characteristics of the NSC. For example, an NSC of the type disclosed herein may exclude allulose, xylose, inulin, chromium, valerian, or cellulose. In an alternative aspect, an NSC of the type disclosed herein excludes allulose, xylose, inulin, chromium, valerian, or cellulose.

In an aspect, an NSC of the type disclosed herein is characterized by a great taste, a reduced cooling effect, a low glycemic index, and exertion of positive effects on the digestive health of the subject ingesting the NSC or an ingestible product comprising the NSC. In an aspect, the NSC exhibits a reduced cooling effect when dissolved in a solvent such as water. Herein, the cooling effect refers to the endothermic solvation of the composition.

In an aspect, the NSC is a component of an ingestible product and exerts a positive effect on the digestive health of the subject consuming the ingestible product. The term “subject,” as used herein, comprises any and all organisms and includes the term “patient.”

In an aspect, the NSC is a prebiotic. Herein, prebiotic refers to non-digestible food ingredients that stimulate the growth and/or activity of beneficial bacterial flora of the gastrointestinal tract. Many digestive disorders are caused by unfavorable alterations in the beneficial bacterial flora of the gastrointestinal tract. The result is a significant disruption of intestinal lining integrity due to a proliferation of pathogens such as E. coli, Clostridium perfringens, H. pylori, etc. This imbalance in microflora, or dysbiosis, is most commonly caused by the use of antibiotics that can wipe out both good and bad bacteria. Other causative factors are GI infections (e.g., traveler's diarrhea), certain drugs such as acid-suppressing medications, chronic mal-digestion, chronic constipation, stress, and diet. A primary role of beneficial gastrointestinal microflora is to help protect the gut lining Once these microflora become imbalanced, the host's immune capabilities become compromised thus leading to more serious digestive disorders such as inflammatory bowel disease (IBD) which include Crohn's disease (CD) and ulcerative colitis (UC). In an aspect, an NSC of the type disclosed herein is a component of a treatment designed to prevent or ameliorate conditions associated with a gastrointestinal disorder such as Crohn's disease or UC.

In an aspect, the NSC may also affect blood glucose and/or fructose levels and have a beneficial effect on insulin sensitivity. These characteristics may be useful in the management of one or more disorders or dysfunction. For example, the NSC and compositions comprising the NSC may facilitate the control of diabetes and/or lower LDL blood cholesterol by interfering with the absorption of dietary cholesterol.

In an aspect, an NSC of the type disclosed herein may be a component of a weight-loss treatment or regimen. The prevalence of obesity among adults in the USA in 2008 was 68% (˜200 million people). Even a modest degree of obesity, particularly if the excess fat is located in the abdomen, increases the risks for type 2 diabetes mellitus, cardiovascular disease, stroke, and some forms of cancer. In an aspect, an NSC of the type disclosed herein and compositions comprising the NSC may be utilized as part of a regimen designed to achieve or maintain a user-determined weight and/or body mass index.

In another aspect, an NSC of the type disclosed herein may be utilized in ingestible products to reduce rapid decreases in blood sugar levels, also termed a “sugar crash.” For example, a typical diet in industrialized nations contains a large amount of simple carbohydrates such as maltodextrin or sugars such as sucrose, fructose, glucose, and dextrose. This type of diet often results in gastrointestinal stress and dehydration due to the high osmotic pressure of the smaller molecules in the stomach leading to dramatic and detrimental swings in blood sugar levels as sugar rapidly enters the blood. The result is the induction of an insulin response that drives down sugar levels, resulting in a sugar crash where blood sugar levels dip below the level prior to consumption of the fuel, thereby exacerbating the effects of non-diabetic hypoglycemia or low blood sugar. In an aspect, an NSC of the type disclosed herein is a component of a regimen designed to reduce the incidence of non-diabetic hypoglycemia in a subject.

In another aspect, the NSC may be a component of a treatment for the prevention or reduction of dental caries. Caries is a multi-factorial disease that involves prolonged colonization of acid-producing bacteria on teeth. Fermentation of dietary carbohydrates by caries leads to a localized drop in pH below a critical value of 5.5, resulting in the demineralization of enamel and potentially damaging underlying tooth structure. If left untreated, caries can lead to the progressive destruction of tooth structure, pain, tooth loss, loss of oral function, as well as have systemic health consequences. In an aspect, an NSC of the type disclosed herein is a component of a regimen designed to reduce the incidence of dental caries in a subject.

In an aspect, an ingestible and/or edible composition comprises, consists essentially of, or consists of an NSC of the type disclosed herein. For example, the NSC may be a component of a carbohydrate-containing food. A subject may be administered the ingestible and/or edible or self-administer the ingestible and/or edible composition.

In an aspect, a subject ingesting a composition comprising, consisting essentially of, or consisting of an NSC of the type disclosed herein experiences a decrease in a postprandial insulin response of from about 10% to about 90%, alternatively from about 25% to about 75%, alternatively greater than about 25%, alternatively greater than about 30%, alternatively greater than about 35%, alternatively greater than about 40%, alternatively greater than about 45%, alternatively greater than about 50%, alternatively greater than about 55%, alternatively greater than about 60%, alternatively greater than about 65%, or alternatively greater than about 70% when compared to a postprandial insulin response obtained following ingestion of an equal amount of sucrose for a time period ranging from about 15 minutes to about 120 minutes. For example, for a time period ranging from about 15 minutes to about 180 minutes subsequent to ingestion of a carbohydrate-containing food comprising an NSC a subject may experience a postprandial insulin response that is reduced by from about 10% to about 90%, alternatively from about 15% to about 80%, alternatively from about 20% to about 75%, alternatively from about 30% to about 75%, or alternatively from about 50% to about 75% when compared to the postprandial insulin response of a subject ingesting an otherwise identical carbohydrate-containing food comprising an equivalent amount of sucrose. In other words the comparison is being made to the response obtained following ingestion of an otherwise identical carbohydrate-containing food that has the sucrose replaced with an NSC of the type disclosed herein.

In an aspect, a subject ingesting a composition comprising, consisting essentially of, or consisting of an NSC of the type disclosed herein experiences a decrease in a postprandial glucose response of from about 10% to about 90%, alternatively from about 25% to about 75%, alternatively greater than about 25%, alternatively greater than about 30%, alternatively greater than about 35%, alternatively greater than about 40%, alternatively greater than about 45%, alternatively greater than about 50%, alternatively greater than about 55%, alternatively greater than about 60%, alternatively greater than about 65%, or alternatively greater than about 70% when compared to a postprandial glucose response obtained following consumption of an equal amount of sucrose for a time period ranging from about 15 minutes to about 120 minutes. For example, for a time period ranging from about 15 minutes to about 180 minutes subsequent to ingestion of a carbohydrate-containing food comprising an NSC a subject may experience a postprandial glucose response that is reduced by from about 10% to about 90%, alternatively from about 15% to about 80%, alternatively from about 20% to about 75%, alternatively from about 30% to about 75%, or alternatively from about 50% to about 75% when compared to the postprandial glucose response of a subject ingesting an otherwise identical carbohydrate-containing food comprising an equivalent amount of sucrose. In other words the comparison is being made to the response obtained following ingestion of an otherwise identical carbohydrate-containing food that has the sucrose replaced with an NSC of the type disclosed herein.

In an aspect, a subject ingesting a composition comprising, consisting essentially of, or consisting of an NSC of the type disclosed herein experiences a decrease in a postprandial fructose response of from about 10% to about 90%, alternatively from about 25% to about 75%, alternatively greater than about 25%, alternatively greater than about 30%, alternatively greater than about 35%, alternatively greater than about 40%, alternatively greater than about 45%, alternatively greater than about 50%, alternatively greater than about 55%, alternatively greater than about 60%, alternatively greater than about 65%, or alternatively greater than about 70% when compared to a postprandial fructose response obtained following consumption of an equal amount of sucrose for a time period ranging from about 15 minutes to about 120 minutes. For example, for a time period ranging from about 15 minutes to about 180 minutes subsequent to ingestion of a carbohydrate-containing food comprising an NSC a subject may experience a postprandial fructose response that is reduced by from about 10% to about 90%, alternatively from about 15% to about 80%, alternatively from about 20% to about 75%, alternatively from about 30% to about 75%, or alternatively from about 50% to about 75% when compared to the postprandial fructose response of a subject ingesting an otherwise identical carbohydrate-containing food comprising an equivalent amount of sucrose. In other words the comparison is being made to the response obtained following ingestion of an otherwise identical carbohydrate-containing food that has the sucrose replaced with an NSC of the type disclosed herein.

In some aspects, the NSC may be administered or self-administered to a subject in the absence of another composition such that the NSC is supplied as a pharmaceutical formulation to a subject. In such aspects the subject may have been diagnosed with or at risk for development of a disorder or dysfunction such as diabetes mellitus type 2, obesity, hypertension, cardiovascular disease, lipidemia, hypertriacylglycerolemia, hypercholesterolemia, chronic kidney disease, or combinations thereof.

Pharmaceutical formulations of the present disclosure may comprise a therapeutically effective amount of an NSC together with a suitable amount of one or more pharmaceutically acceptable vehicles so as to provide a composition for proper administration to a subject. For example, an NSC may be incorporated into a pharmaceutical formulation to be administered orally. Oral administration of such pharmaceutical formulations may result in uptake of an NSC of the type disclosed herein throughout the intestine and entry into the systemic circulation. Such oral compositions may be prepared in any pharmaceutically acceptable manner and comprise an NSC and at least one pharmaceutically acceptable vehicle, so as to provide an appropriate form for administration to a subject.

An NSC of the type disclosed herein may be incorporated into pharmaceutical compositions to be administered by any other appropriate route of administration. Pharmaceutical formulations provided by the present disclosure may take the form of solutions, suspensions, emulsion, pills, buccal tablet, sub-lingual tablet, orally-disintegrating tablet, liquid, suspension, powder, capsules containing liquids, powders, sustained-release formulations suspensions, or any other form suitable for administration to or by a subject.

Pharmaceutical formulations provided by the present disclosure may be formulated in a unit dosage form. A unit dosage form refers to a physically discrete unit suitable as a unitary dose for patients undergoing treatment, with each unit containing a predetermined quantity of an NSC calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to or by a subject at a single point in time or during a time interval.

In an aspect, the amount of NSC provided in any dosage form ranges from about 1 mg to about 1000 mg, alternatively from about 10 mg to about 1000 mg, alternatively from about 50 mg to about 500 mg, alternatively about 25 mg, alternatively about 50 mg, alternatively about 75 mg, alternatively about 100 mg, alternatively about 125 mg, alternatively about 150 mg, alternatively about 175 mg, alternatively about 200 mg, alternatively about 225 mg, alternatively about 250 mg, alternatively about 275 mg, alternatively about 300 mg, alternatively about 325 mg, alternatively about 350 mg, alternatively about 375 mg, alternatively about 400 mg, alternatively about 425 mg, alternatively about 450 mg, alternatively about 475 mg, alternatively about 500 mg, alternatively about 525 mg, alternatively about 550 mg, alternatively about 575 mg, alternatively about 600 mg, alternatively about 625 mg, alternatively about 650 mg, alternatively about 675 mg, alternatively about 700 mg, alternatively about 725 mg, alternatively about 750 mg, alternatively about 775 mg, alternatively about 800 mg, alternatively about 825 mg, alternatively about 850 mg, alternatively about 875 mg, alternatively about 900 mg, alternatively about 925 mg, alternatively about 950 mg, alternatively about 975 mg, alternatively about 1000 mg, alternatively greater than about 100 mg, alternatively greater than about 250 mg, or alternatively greater than about 500 mg.

In each aspect of the utilization of an NSC of the type disclosed herein as a component in the treatment of a disease or dysfunction, it is contemplated the subject will be administered and/or supplied an ingestible product comprising the NSC. The NSC may partially or fully replace sugar in said product and thus the subject is given a reduced calorie, ingestible material as a component of the treatment. It is contemplated that a treatment of the type disclosed herein may also comprise conventional methodologies. For example, for obesity the treatment in addition to comprising ingestion of the NSC may further comprise exercise and other activities or compositions that function to promote weight loss. Thus, in an aspect, a methodology of the type disclosed herein further comprises the ingestion of the NSC in conjunction with one or more conventional therapies for addressing the particular disorder or dysfunction. For example, ingestion of the NSC may be a component of a weight loss regimen that can include a reduced fat and/or caloric diet, exercise, weight-reducing medication and/or weight reducing surgery. In an alternative aspect, ingestion of the NSC may be a component of diet specialized to treat one or more disorders or dysfunctions such as a heart-healthy diet, a diabetic diet, and the like.

In some aspects an NSC is a component of a carbohydrate containing food that is ingested by a subject. In an alternative aspect, the NSC is provided in a dosage form that is ingested by the subject prior to, concurrent with or post-ingestion of a carbohydrate-containing food. In aspects where the NSC is ingested prior to or post-ingestion of a carbohydrate-containing food, the interval between ingestion of the carbohydrate-containing food and the NSC may range from about 1 minute to about 180 minutes, alternatively from about 5 minutes to about 120 minutes, or alternatively form about 30 minutes to about 120 minutes.

In an aspect, the present disclosure comprises a blend of an NSC and an artificial sweetener. The blend may contain a ratio of NSC:artificial sweetener ranging from 1:10 to 10:1, alternatively from 3:7 to 7:3, or alternatively 1:1. Examples of artificial sweeteners suitable for use in the blend include without limitation acesulfame potassium, aspartame, neotame, sucralose, advantame, and combinations thereof.

While aspects of the present disclosure have been shown and described, modifications thereof can be made by one skilled in the art without departing from the spirit and teachings of the disclosure. The aspects described herein are exemplary only, and are not intended to be limiting. Many variations and modifications of the disclosure are possible and are within the scope of the invention. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present invention. Thus, the claims are a further description and are an addition to the preferred aspects of the present invention. The discussion of a reference in the Background is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application.

Unless indicated otherwise, when a range of any type is disclosed or claimed it is intended to disclose or claim individually each possible number that such a range could reasonably encompass, including any sub-ranges encompassed therein. When describing a range of measurements every possible number that such a range could reasonably encompass can, for example, refer to values within the range with one significant digit more than is present in the end points of a range. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure.

### ADDITIONAL DISCLOSURE

A first aspect which is a method of attenuating a postprandial insulin response comprising administering to a subject a composition comprising at least one sugar alcohol, stevia, monk fruit, a rheology modifier, and tagatose wherein a subject ingesting a carbohydrate-containing food comprising the composition experiences a decrease in the postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

A second aspect which is the method of the first aspect wherein the subject ingesting the carbohydrate-containing food comprising the composition experiences a decrease in the postprandial insulin response of from about 20% to about 75% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

A third aspect which is the method of any of the first through second aspects wherein the subject ingesting a carbohydrate-containing food comprising the composition experiences a decrease in a postprandial glucose response of from about 10% to about 90% for a time period ranging from about 15 minutes to about 180 minutes subsequent to ingestion of the carbohydrate-containing food when compared to the postprandial glucose response of a subject ingesting an otherwise identical carbohydrate-containing food comprising an equivalent amount of sucrose.

A fourth aspect which is the method of any of the first through third aspects wherein the subject ingesting a carbohydrate-containing food comprising the composition experiences a decrease in a postprandial glucose response of from about 20% to about 75% for a time period ranging from about 15 minutes to about 180 minutes subsequent to ingestion of the carbohydrate-containing food when compared to the postprandial glucose response of a subject ingesting an otherwise identical carbohydrate-containing food comprising an equivalent amount of sucrose.

A fifth aspect which is the method of any of the first through fourth aspects wherein the subject ingesting a carbohydrate-containing food comprising the composition experiences a decrease in a postprandial fructose response of from about 10% to about 90% for a time period ranging from about 15 minutes to about 180 minutes subsequent to ingestion of the carbohydrate-containing food when compared to the postprandial fructose response of a subject ingesting an otherwise identical carbohydrate-containing food comprising an equivalent amount of sucrose.

A sixth aspect which is the method of any of the first through fifth aspects wherein the subject ingesting a carbohydrate-containing food comprising the composition experiences a decrease in a postprandial fructose response of from about 20% to about 75% for a time period ranging from about 15 minutes to about 180 minutes subsequent to ingestion of the carbohydrate-containing food when compared to the postprandial fructose response of a subject ingesting an otherwise identical carbohydrate-containing food comprising an equivalent amount of sucrose.

A seventh aspect which is the method of any of the first through sixth aspects wherein the subject is a human.

An eighth aspect which is the method of any of the first through seventh aspects wherein the subject is diagnosed with diabetes mellitus type 2, obesity, hypertension, cardiovascular disease, lipidemia, hypertriacylglycerolemia, hypercholesterolemia, chronic kidney disease, or combinations thereof.

A ninth aspect which is the method of any of the first through eighth aspects wherein the subject is administered from about 10 mg to about 1000 mg of the composition.

A tenth aspect which is the method of any of the first through ninth aspects wherein the subject is administered from about 50 mg to about 500 mg of the composition.

An eleventh aspect which is the method of any of the first through tenth aspects wherein the composition is self-administered.

A twelfth aspect which is the method of any of the first through eleventh aspects wherein the composition is administered at least once daily.

A thirteenth aspect which is the method of the eighth aspect wherein the composition is administered at least once daily.

A fourteenth aspect which is the method of the ninth aspect wherein the composition is administered at least once daily.

A fifteenth aspect which is the method of any of the first through fourteenth aspects wherein the composition excludes allulose, xylose, inulin, chromium, valerian, or cellulose.

A sixteenth aspect which is a method of attenuating a postprandial glucose response comprising administering to a subject a composition comprising at least one sugar alcohol, stevia, monk fruit, a rheology modifier, and tagatose wherein a subject ingesting (i) a carbohydrate-containing food and (ii) the composition experiences a decrease in a postprandial insulin response of from about 10% to about 90% for a time period ranging from about 15 minutes to about 180 minutes subsequent to ingestion of the composition when compared to the postprandial insulin response of a subject ingesting an otherwise identical carbohydrate-containing food comprising an equivalent amount of sucrose.

A seventeenth aspect which is the method of the sixteenth aspect wherein the composition is provided as a dosage form selected from the group consisting of pills, buccal tablet, sub-lingual tablet, orally-disintegrating tablet, liquid, suspension, and powder.

An eighteenth aspect which is the method of any of the sixteenth through seventeenth aspects wherein the composition excludes allulose, xylose, inulin, chromium, valerian, or cellulose.

A nineteenth aspect which is the method of any of the sixteenth through eighteenth aspects wherein the subject ingests from about 10 mg to about 1000 mg of the composition.

A twentieth aspect which is the method of any of the sixteenth through nineteenth aspect wherein a subject experiences a decrease in a postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

A twenty-first aspect which is the method of any of the sixteenth through twentieth aspects wherein a subject experiences a decrease in a postprandial fructose response of from about 10% to about 90% for a time period ranging from about 15 minutes to about 180 minutes subsequent to ingestion of the carbohydrate-containing food when compared to the postprandial fructose response of a subject ingesting an otherwise identical carbohydrate-containing food comprising an equivalent amount of sucrose.

### EXAMPLES

The present disclosure is further illustrated by the following example, which is not to be construed in any way as imposing limitations upon the scope thereof. On the contrary, it is to be clearly understood that resort can be had to various other aspects, aspects, modifications, and equivalents thereof which, after reading the description herein, can suggest themselves to one of ordinary skill in the art without departing from the spirit of the present disclosure or the scope of the appended claims.

The data and descriptions provided in the following example are given to show particular aspects and aspects of the subject matter disclosed, and to demonstrate a number of the practices and advantages thereof. The example is given as a more detailed demonstration of some of the aspects and aspect described herein and is not intended to limit the disclosure or claims in any manner.

### Example 1

The blood glucose response to NSCs of the type disclosed herein was compared to that of table sugar. Specifically, an NSC containing erythritol, tagatose, maltitol, monkfruit, xanthum gum and natural flavors was formulated. Subjects' baseline blood glucose level was determined and set as 100% at time 0. The subjects then ingested a sufficient amount of NSC to provide 50 grams of carbohydrates and the blood glucose levels were determined at periodic intervals for up to 120 minutes after ingestion. A plot of the blood glucose level increase as a percentage above baseline for an NSC of the type disclosed herein is presented in FIG. 2. The blood glucose level change following the ingestion of sugar in an amount to provide 50 grams of carbohydrates is also shown in FIG. 2.

The results demonstrate an NSC of the type disclosed herein results in minimal changes in the blood glucose levels over the measured time period in comparison to table sugar.

### Example 2

The effect on the GI response for different NSC formulations was assessed. Four formulations, designated F1-F4, containing tagatose, monkfruit, stevia extract, erythritol, xanthan gum and either maltitol or lactitol were prepared as indicated in Table 1. The glycemic index for each formulation was calculated based on the glycemic index for the individual components taking into account the weight percent of the component in the formulation. Monkfruit, xanthan gum, and stevia extract have a glycemic index of 0, erythritol has a glycemic index of 1 while tagatose has a glycemic index of 3. Maltitol has a glycemic index of 35 while lactitol has a glycemic index of 5. Fifteen subjects were evaluated and the GI calculated as described previously herein.

TABLE 1

| Sample | Maltitol | Lactitol | Expected GI | Observed GI |
|---|---|---|---|---|
| F1 | YES | NO | 7.21 | 4.41 |
| F2 | NO | YES | 2.27 | 6.43 |
| F3 | YES | YES | 4.74 | 3.03 |
| F4 | NO | NO | 3.00 | 2.09 |
| SUGAR | NO | NO | 65 | 65 |

The results demonstrate that unexpectedly, maltitol despite having a much higher glycemic index than lactitol, 35 and 5 respectively; produced NSCs having a lower than expected overall glycemic index.

### Example 3

The effect of an NSC on the blood glucose level attenuation in the presence of a CCF was investigated. Specifically, a cake mix was prepared containing the ingredients listed in Table 2.

TABLE 2

| | | | | % in | |
|---|---|---|---|---|---|
| | Mass | | Glycemic | dry mass | g per |
| Ingredient | (Quantity) | | Index | (est.) | cupcake |
| 112 g flour | 112 | g | 71 extra carbs | 33.4% | 14 |
| 100 g sweetener | 100 | g | 65 for sugar | 29.8% | 12.5 |
| or sugar | | | <5 for NSC | | |
| ½ tsp baking | 2.84 | g | 0 | 0.85% | 0.355 |
| powder | (½ | tsp) | | | |
| ½ tsp baking soda | 2.84 | g | 0 | 0.85% | 0.355 |
| | (½ | tsp) | | | |
| ¼ tsp salt | 1.42 | g | 0 | 0.42% | 0.1775 |
| | (¼ | tsp) | | | |
| 60 ml buttermilk | 62.16 | g | 31 extra carbs | 18.53% | 7.77 |
| | (60 | ml) | | | |
| 70 g unsalted | 70 | g | 0 | 20.86% | 8.75 |
| butter | | | | | |
| 1 extra large egg | 56 | g | 0 | 16.69% | 7 |
| 1½ tsp vanilla | 6.5 | g | 0 | 1.94% | 0.81 |
| extract | | | | | |

| | |
|---|---|
| Total 413.7 g wet, 335.51 g dry (dry has | Total extra |
| a 19% moisture loss) Yield 8 cupcakes | carbs = 34.27 g |

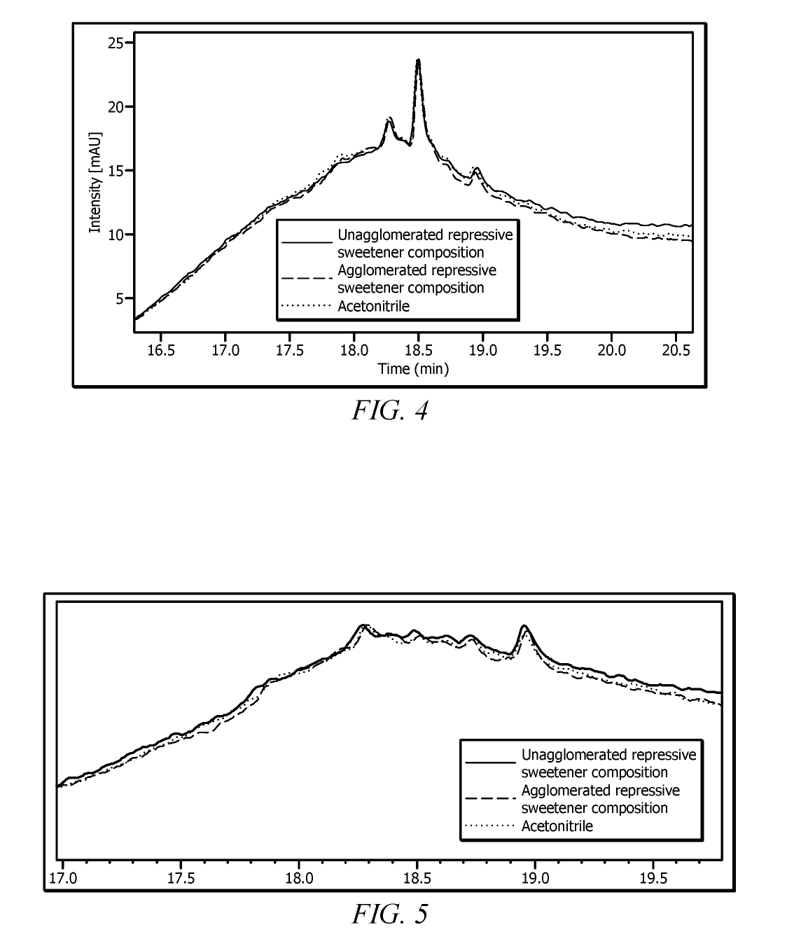

The dry mix contained 34.27 g of extra carbs. Cupcakes were prepared using either sugar, the F1 NSC from example 2 which had maltitol, or the F3 NSC from example 2 which contained both maltitol and lacitol. A plot of the percentage increase for subjects ingesting the cupcakes as a function of time is presented in FIG. 4. The data demonstrate that cupcakes prepared with the NSC F1 (i.e., containing maltitol) resulted in a lower blood glucose level increase over the baseline when compared to ingestion of cupcakes prepared with either sugar or the NSC F3 which had a combination of maltitol and lactitol. The data also demonstrate the suppressive effect of NSC F1 (i.e., containing maltitol) persists in the presence of other carbohydrate-containing ingredients.

### Example 4

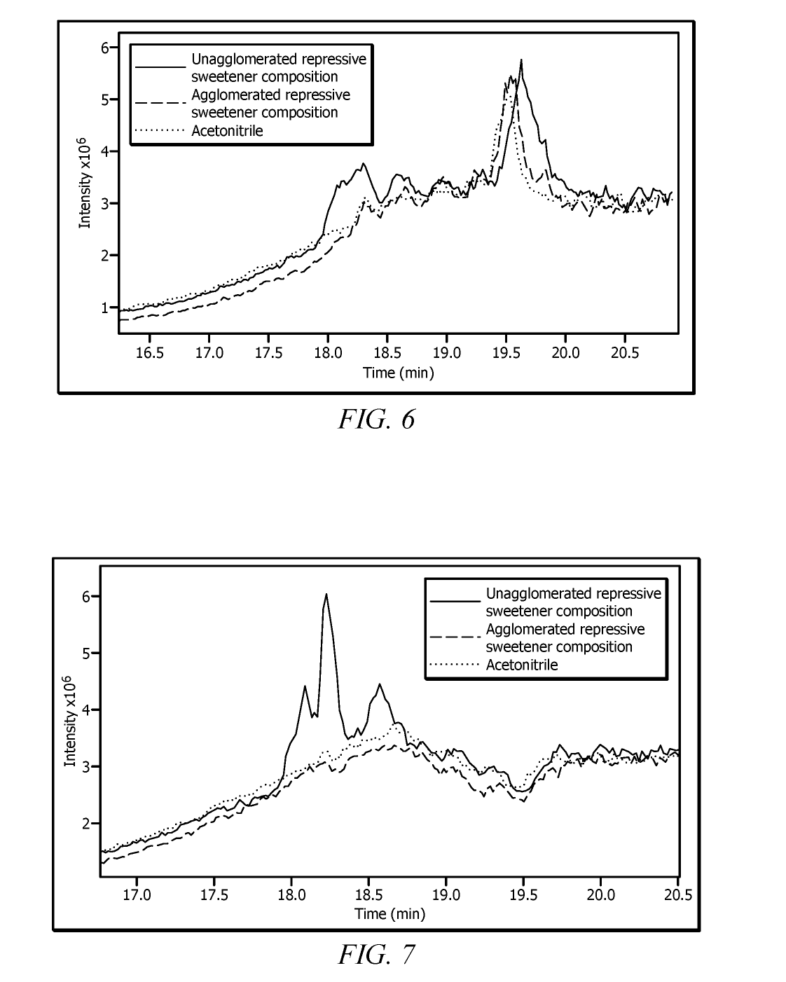

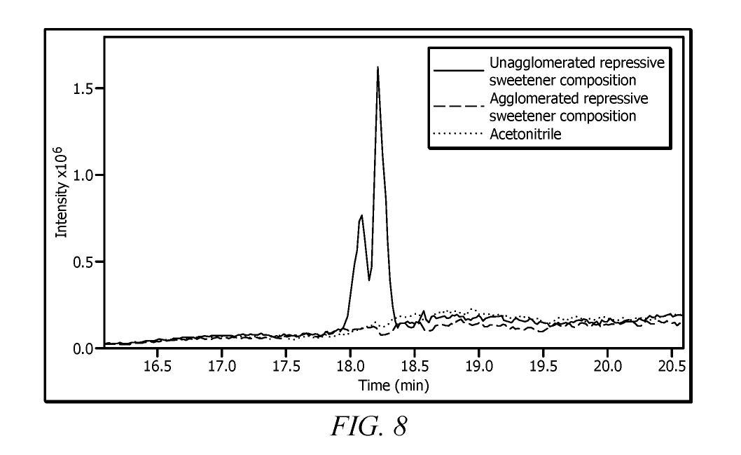

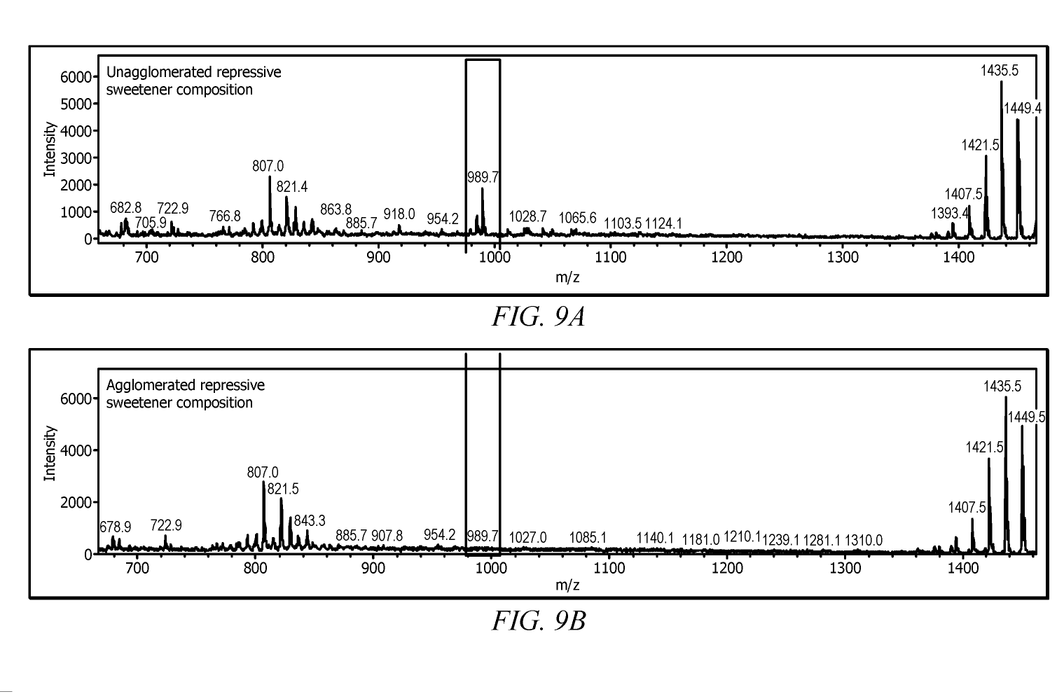

The ESI mass spectrum for an NSC was obtained. Specifically, each sample was weighed out (˜10 mg) and dissolved to a final concentration 10 mg/ml. The samples were then extracted using acetonitrile (ACN). Fisher Optima LCMS grade was used. The samples which were not fully soluble at that concentration were vortexed 15 seconds, waited, and vortexed again. Aliquots (100 μl) were removed for LCMS analysis and 8 μl injected onto an Agilent ZORBAX SB-C18 (2.1×100 mm) column. Solvent A was 5% ACN/94% H₂O/1% acetic acid while solvent B was Methanol 100%. The column was run at a flow rate of 200 μl/min using two gradients: (1) a slow gradient 5-60% B in 10 min, then to 100% B in 5 min, 3 min hold and (2) a steeper gradient 5-100% B in 15 min, 3 min hold (18 min total). Sample analysis was carried out in UV using the full spectrum 190-400 nm, and 215, 275 and 290 nm single wavelength chromatographic trace while the mass spectrum utilized a gentle source with a sensitivity optimized for m/z around 800 and the analysis was conducted in both negative and positive ionization mode. FIGS. 4 and 5 are overlays of the UV spectrum for the unagglomerated NSC (red), the agglomerated NSC (blue) and acetonitrile (green). Analysis of FIGS. 4 and 5 suggests there is little difference between the agglomerated and unagglomerated samples. Mass spectrometry of the samples in positive mode demonstrates a difference between the unagglomerated and agglomerated samples, FIGS. 6 and 7. Similar trends were observed when using the negative ESI mode, FIG. 8. A comparison of the LCMS traces between the unagglomerated and agglomerated NCS is shown in FIG. 9 which demonstrates a clear difference in the m/z range of 967 to 989.

### Example 5

A sweetener composition of the type disclosed herein (i.e., an NSC) will be compared to table sugar and other commercially available sweetners using 6 differentiating tests carried out on at least 10 members of the lay public, lacking specialized knowledge in sweeteners:

### 1) The Sugar Similarity Test

An amount of refined white sugar consisting of typical comparable granule size and fine content is placed into a bowl and compared one by one with other sugar substitutes, including the Natural Sweetener described herein. 10 members of the lay public, lacking specialized knowledge in sweeteners, are asked to compare the sugar versus the sugar substitute in the bowl and identify which is sugar and which is the sugar substitute based on appearance, smell and taste within a 1 minute period. A sweetener is considered to pass the test and be enough like sugar when 80% of the people testing fail to differentiate the sugar versus the sugar substitute based on appearance, smell and dipping their fingertip into the sugar or sugar substitute to taste.

### 2) The Yellow Cake-Bake Test

A cake that when baked has a light yellow hue is made from the following ingredients: 120 g flour, 150 g sugar or sweetener, ½ teaspoon baking powder, ½ teaspoon baking soda, ½ teaspoon salt, 65 ml buttermilk, 30 g vegetable oil, 30 g unsalted butter (softened), 1 extra large egg, 1½ teaspoon vanilla extract. The oven is pre-heat to 300 F, flour is sifted, sugar or sweetener is added with baking powder, baking soda and salt into a large standing mixer bowl. In a separate bowl the egg, vanilla extract, buttermilk and vegetable oil are whisked together. Using the whisk attachment on the standing mixer the butter is added to the dry ingredients and mixed well on a low speed until well mixed. The liquid mixture is slowly added to the dry ingredients and once combined turn the mixer up to full speed and whisk for one minute until all the ingredients are homogeneous. The mixture is divided between 8 cupcake cases spaced out evenly in a muffin pan and place into the oven for 10 minutes. The pan is then turned and baked for a further 10 minutes. A skewer is inserted into the cakes to ensure they are done; the skewer coming out clean if the cakes are done.

A sweetener is considered to pass the test and be enough like sugar when a) 80% of the people testing fail to differentiate the cake made with sugar versus the cake made with sugar substitute based on appearance, smell, taste and mouth-feel within the same day of being baked b) 80% of the people testing fail to differentiate the cake made with sugar versus the cake made with sugar substitute based on appearance, smell, taste and mouth-feel 3 days after being stored in an open container (i.e. a moisture retention test).

### 3) The Caramelization Test

1 cup of caramelized sugar or 1 cup of sweetener is mixed with ½ cup of water and swirled to mix until it browns. A sweetener is considered to pass the test and be enough like sugar when it browns within 25% of the time when the sugar browns.

### 4) The Chocolate Bar Test

Chocolate bars are prepared by blending 200 g melted cocoa liquor, 250 g melted cocoa butter into running bench-top chocolate melangeur (“Santha” brand chocolate melangeur), once well blended 1 teaspoon of soya lecithin, 250 g dry powdered whole milk powder, 0.06% ground vanilla bean and 350 g sugar or sweetener and added gradually and then allowed to mélange for 1.5 days. The mix is then tempered, molded and cooled. A sweetener is considered to pass the test and be enough like sugar when 80% of the people testing fail to differentiate the chocolate bar made with sugar versus the sugar substitute made with sugar based on appearance, smell and mouth-feel. This test is designed to screen out sweeteners that produce a strong endothermic or cooling sensation when the chocolate is melted on the tongue.

### 5) The Tea Test

Iced tea is prepared using established methods and sugar or sweetener is used at twice the recommended concentration. A sweetener is considered to pass the test and be enough like sugar when 80% of the people testing fail to differentiate the Iced tea made with sugar versus Iced tea made with sugar substitute based on appearance, smell, taste and lack of unusual after-taste.

### 6) A Glycemic Response Test

Under an Institutional Review Board approved, controlled clinical trial, subjects undergo a pre-test fast for at least 20 hours and then consume 30 grams of glucose, or an equal amount of sweetener in water. Blood glucose measurements are then made every 15 minutes for 90 minutes and the blood glucose response is recorded. A sweetener is considered to pass the test when then blood glucose response is less than 5% of glucose.

### 7) A Repression Test

Under controlled study scenario subjects fast, then consume a product (e.g. a cupcake) that contains other nutritive carbohydrates and the glycemic response is assessed by established methods. The glycemic response to the nutritive carbohydrates is compared to the glycemic response to the nutritive carbohydrates in the presence of the sweetener or sugar. A sweetener is considered to pass the repression test when then blood glucose response is much lower than expected for the combination of

The results of these tests are summarized in Table 3.

TABLE 3

| | | | A mix of | | Erythritol, | | Dextrose, | | |
|---|---|---|---|---|---|---|---|---|---|
| | | | sucralose, | | oligosac- | | Acesulfame-K, | | |
| | | Natural | dextrose | Rebaudioside | charides, | Saccharin, | Cream of Tartar, | Dextrose, | |
| | | Sweetener | and malto- | A, erythritol, | natural | dextrose, | Calcium | Rebaudioside | Dextrose, |
| | | (described | dextrin | natural | flavors | cream of | Silicate, | A, Natural | maltodextrin, |
| | Sugar | herein) | (Splenda) | flavors | (Swerve) | tartar | Natural Flavors. | Flavors | aspartame |
| Sugar similarity | PASS | PASS | FAIL | FAIL | FAIL | FAIL | FAIL | FAIL | FAIL |
| test | | | | | | | | | |
| Yellow cake- | PASS | PASS | FAIL | FAIL | FAIL | FAIL | FAIL | FAIL | FAIL |
| bake test | | | | | | | | | |
| Caramelization | PASS | PASS | | | | | | | |
| test | | | | | | | | | |
| Chocolate | PASS | PASS | FAIL | FAIL- | FAIL- | FAIL- | FAIL- | FAIL- | FAIL- |
| bar test | | | | AFTER | STRONG | AFTER | AFTER | AFTER | AFTER |
| | | | | TASTE | COOLING | TASTE | TASTE | TASTE | TASTE |
| | | | | | SENSATION | | | | |
| Tea test | PASS | PASS | PASS | FAIL- | FAIL- | FAIL- | FAIL- | FAIL- | FAIL- |
| | | | | VANILLA | COOLING | AFTER | AFTER | AFTER | AFTER |
| | | | | AFTER | AFTER | TASTE | TASTE | TASTE | TASTE |
| | | | | TASTE | TASTE | | | | |
| Glycemic | FAIL | PASS | CONTAINS | PASS | PASS | CONTAINS | CONTAINS | CONTAINS | CONTAINS |
| test | | | DEXTROSE | | | DEXTROSE | DEXTROSE | DEXTROSE | DEXTROSE |
| Repression | FAIL | PASS | FAIL | FAIL | FAIL | FAIL | FAIL | FAIL | FAIL |
| test | | | | | | | | | |

| | | | | Tagatose, | | | | |
|---|---|---|---|---|---|---|---|---|
| | Liquid drops | | | erythritol | | | | |
| | made from a | | | plus stevia, | | | | |
| | stevia leaf | inulin, | Dextrose, | monkfruit | | | | |
| | extraction | stevia, | monkfruit | or stevia and | | Isomaltooligo- | | |
| | process | silica | extract | monkfruit | Erythritol | saccahrides | Tagatose | Allulose |
| Sugar similarity | FAIL | FAIL | FAIL | PASS | FAIL | FAIL | PARTIAL | PARTIAL |
| test | | | | | | | (LESS | (LESS |
| | | | | | | | SWEET) | SWEET) |
| Yellow cake- | FAIL | FAIL | FAIL | FAIL - | FAIL | FAIL | FAIL- | PASS |
| bake test | | | | DOES NOT | | | TOO DARK | |
| | | | | RETAIN | | | BROWNS | |
| | | | | MOISTURE | | | TOO FAST | |
| Caramelization | | | | | | | FAIL- | |
| test | | | | | | | TOO DARK | |
| | | | | | | | BROWNS | |
| | | | | | | | TOO FAST | |
| Chocolate | FAIL- | FAIL- | FAIL- | PASS | FAIL- | FAIL- | PARTIAL | PARTIAL |
| bar test | AFTER | AFTER | TASTE | | STRONG | RHEOLOGY | (LESS | (LESS |
| | TASTE | TASTE | | | COOLING | | SWEET) | SWEET) |
| | | | | | SENSATION | | | |
| Tea test | FAIL- | FAIL-NOT | FAIL-NOT | PASS | FAIL- | FAIL-NOT | PARTIAL | PARTIAL |
| | AFTER | SWEET | SWEET | | AFTER | SWEET | (LESS | (LESS |
| | TASTE | ENOUGH | ENOUGH | | TASTE | ENOUGH | SWEET) | SWEET) |
| Glycemic | PASS | PASS | CONTAINS | PASS | PASS | FAIL | PASS | PASS |
| test | | | DEXTROSE | | | | | |
| Repression | FAIL | FAIL | FAIL | FAIL | FAIL | FAIL | FAIL | PASS |
| test | | | | | | | | |

### Example 6

An acute randomized controlled trial was carried out to investigate the effects of an NSC of the type disclosed herein on postprandial insulin, glucose and fructose responses.

Subjects (N=15) men and non-pregnant, non-lactating women, 18-75 years of age each with a BMI of 18-40 kg/m² inclusive and normal glycemia and (i) fasting serum glucose <126 mg/dl (<7.0 mmol/l) (or in whole blood ≦6.1 mmol/L); (ii) blood pressure <130/85; (iii) no prescription drugs within 1 month of the first study visit after screening except for the birth control pill, lipid lowering medications or small doses of anxiolytics/sedatives to assist sleeping; and (iv) no major illness or surgery requiring hospitalization within 3 months of the first study visit after screening. Subjects could be excluded from the study for one or more of the following: (i) failure to meet all the inclusion criteria; (ii) previous bariatric procedure; (iii) presence of chronic disease such as type-1 or type-2 diabetes mellitus (iv) any history of diabetes; (v) presence of gastro-intestinal disorder such as Crohn's disease, coeliac disease, irritable bowel syndrome; (vi) strong dislike of or intolerance to sweetened beverages; (vii) use of antibiotics within 15 days prior to inclusion in the study; (viii) illness or medication within 48 hours of study visits; (ix) does not understand English; or (x) the presence of any condition, illness or drug use, which in the opinion of the study professional, increases the risk to the subject or to others or may affect the results.

Number of subjects: Using the t-distribution and assuming an average CV of within-individual variation of 2-hour incremental AUC values of 23%, n=15 subjects has 80% power to detect a 25% difference in 2-hour incremental glucose AUC with 2-tailed p<0.05.

Subjects arrived after 10-14 hr overnight fast during which only water was allowed. The day prior to each visit, participants were encouraged to consume the same evening meal and abstain from tobacco products for at least 1 hour prior to the test. For 24-hr prior to each visit, participants avoided unusual levels of exercise. Participants refrained from taking any medication except those which were listed in the inclusion criteria.

Participants were also asked not take any medication including acetaminophen in the morning of testing. If any subject was not feeling well or had not complied with the preceding experimental conditions, the test was not carried out and was rescheduled for another day.

After being weighed, 2 fasting blood samples at 5 min intervals were collected; subjects started to consume a test beverage (sweetener mixed with water) which was consumed in its entirety within 5 min. A timer was started at the first sip of the beverage, and further capillary blood samples were obtained at t=15, 30, 45, 60, 90 and 120±2 min, for the determination of glucose, insulin and fructose concentrations. Blood samples were collected into 2 separate vials: one (2-3 drops blood) for glucose analysis and the other (8-10 drops of blood) for insulin and fructose.

The test foods consisted of 2 isovolumetric beverages: 30 g sucrose control and 30 g of sweetener blend dissolved in 500 ml water. The sucrose test meal acted as the control. Test meals were administered in a randomized order.

Blood collection tubes were labeled with the anonymized subjects ID, visit date, test meal code and time of collection. Following blood collection, the tubes containing blood for glucose analysis were rotated to mix the blood with the anti-coagulant (sodium fluoride potassium oxalate) and then placed in a refrigerator until the last blood sample in the set has been collected. All tubes were placed in a −20° C. freezer until analysis which was performed within 5 days. Glucose analysis was performed using an YSI model 2300 STAT analyzer (Yellow Springs, Ohio) with an intra CV of less than 2.0%. The microvette tubes containing blood for insulin and fructose were left at room temperature for at least 20 minutes to allow the blood to clot, centrifuged at room temperature for 10 minutes at 3,000 rpm and the serum transferred to labeled polypropylene tubes and stored at −20° C. prior to analysis of insulin. Serum insulin levels were measured using the Human Insulin EIA Kit (Alpco Diagnostics, catalog #80-INSHU-E10.11). The manufacturers' intra CV is 10.3%, 3.2%, 5.1% for low, medium to high insulin levels. The manufacturers inter assay our lab the intra CV is 7%. Serum fructose levels were measured using the EnzyChrom™ Fructose Assay Kit (EFRU-100, BioAssay Systems, Hayward Calif.).

Data are presented as mean and standard error of the mean (SEM) values. The mean glucose, insulin or fructose concentration in the 2 fasting blood samples was used as the fasting concentration for the purposes of calculating incremental areas under the blood glucose, serum insulin and fructose response curves (IAUC), ignoring area below fasting. Glucose, insulin and fructose concentrations, glucose, insulin and fructose IAUC (from 0 to 120 minutes) will be subjected to repeated measures ANOVA. After demonstration of significant heterogeneity, the significance of differences among individual means were determined using Tukey's method. In addition, difference at individual time points for glucose, insulin and fructose were assessed. Differences were considered to be statistically significant if 2-tailed p<0.05.

Eighteen subjects were screened of which 3 were ineligible according to the inclusion/exclusion criteria. A total of and fifteen subjects were therefore enrolled into the study (9M;6F, age: 29±10 y, BMI: 26.3±2.7 kg/m²). The individual details are shown in Table 4.

TABLE 4

| | | | Age | Height | | Weight | | BMI | Blood Pressure | | FBG |
|---|---|---|---|---|---|---|---|---|---|---|---|
| ID | Sex | Ethnicity | (yrs) | (cm) | (in) | (kg) | (lb) | (kg/m²) | Systolic | Diastolic | (mmol/L) |
| 1 | M | Chinese | 54 | 167.4 | 65.3 | 78.1 | 171.8 | 27.9 | 113 | 69 | 4.50 |
| 2 | M | Caucasian | 28 | 183.0 | 71.4 | 67.1 | 191.6 | 26.0 | 126 | 71 | 4.53 |
| 3 | F | Caucasian | 48 | 168.4 | 65.7 | 74.3 | 163.5 | 26.2 | 93 | 54 | 4.17 |
| 5 | F | South East Asian | 36 | 161.1 | 62.8 | 60.2 | 132.4 | 23.2 | 113 | 71 | 4.21 |
| 4 | M | Chinese | 20 | 156.0 | 64.7 | 59.0 | 129.3 | 21.4 | 113 | 61 | 3.90 |
| 8 | F | Latin American | 26 | 169.0 | 65.9 | 67.3 | 148.1 | 23.6 | 109 | 62 | 4.69 |
| 7 | F | Latin American | 28 | 161.5 | 63.0 | 81.8 | 180.0 | 31.4 | 108 | 67 | 4.66 |
| 6 | M | Caucasian | 20 | 178.0 | 69.4 | 76.5 | 168.3 | 24.1 | 120 | 67 | 4.20 |
| 9 | F | Chinese | 29 | 164.7 | 64.2 | 72.4 | 159.3 | 26.7 | 106 | 57 | 4.29 |
| 10 | M | Caucasian | 18 | 176.8 | 69.0 | 77.0 | 169.4 | 24.6 | 125 | 64 | 4.24 |
| 12 | M | Black | 29 | 181.5 | 70.8 | 90.5 | 199.1 | 27.5 | 126 | 70 | 4.30 |
| 11 | M | Chinese | 24 | 166.2 | 64.8 | 72.1 | 158.6 | 26.1 | 107 | 58 | 3.96 |
| 13 | F | Arab/West Asian | 20 | 165.0 | 64.4 | 82.3 | 181.1 | 30.2 | 105 | 75 | 4.77 |
| 14 | M | Caucasian | 24 | 184.0 | 71.8 | 96.5 | 212.3 | 28.5 | 113 | 60 | 3.85 |
| 15 | M | Caucasian | 32 | 165.5 | 64.5 | 72.3 | 159.1 | 26.4 | 112 | 55 | 4.78 |

| | | | | | | | | | |
|---|---|---|---|---|---|---|---|---|---|
| MEAN | 29.1 | 170.5 | 66.5 | 76.5 | 168.3 | 26.3 | 112.6 | 64.1 | 4.34 |
| SD | 10.2 | 7.9 | 3.1 | 10.3 | 22.7 | 2.7 | 8.9 | 6.5 | 0.31 |

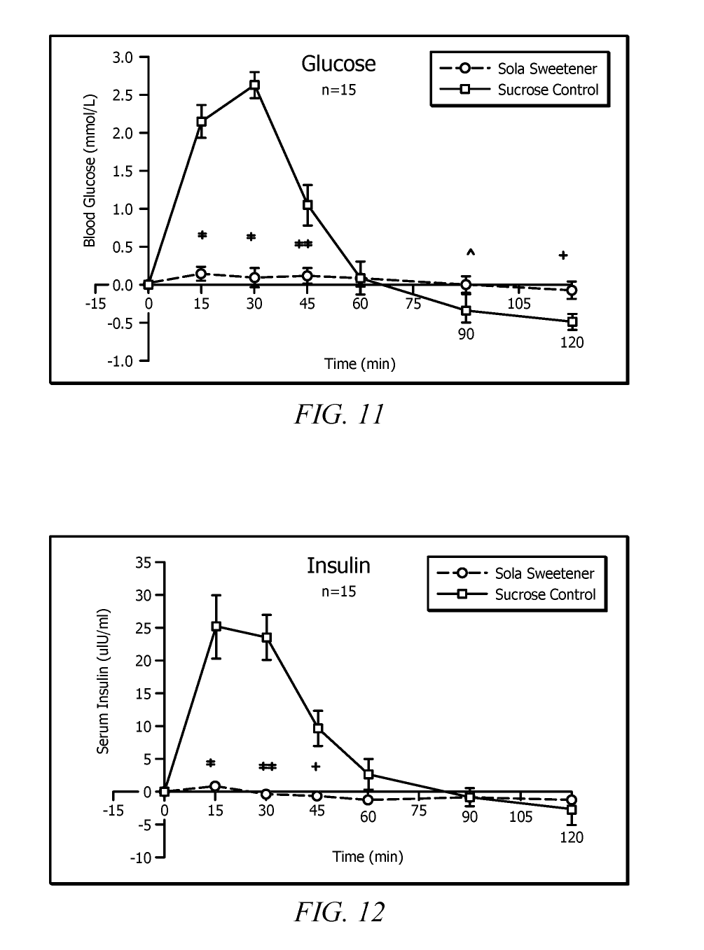

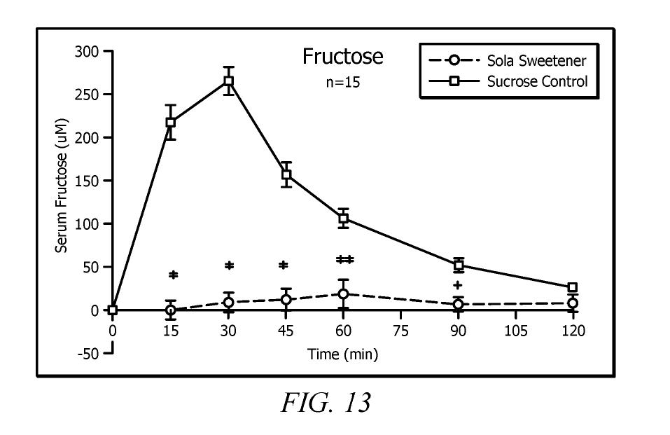

The postprandial blood glucose, insulin, and fructose responses are plotted in FIGS. 11, 12, and 13 respectively. At 15, 30, 45, 90 and 120 min blood glucose, insulin and fructose levels were significantly lower after ingestion of an NSC of the type disclosed herein than after the sucrose control. In the case of glucose, at 90 and 120 min the blood glucose levels were significantly higher after the NSC Sweetener than after the sucrose control (p<0.04).

Adverse events reported following ingestion of the meals containing either an NSC of the type disclosed herein or sucrose included bloating, belching, nausea, diarrhea and flatulence. Each issue was scored and record for the time period indicated in Table 5 which provides the symptom score (mm) for: Bloating, Belching, Nausea, Diarrhea and Flatulence at 0 min, 120 min, before Dinner (D) and before breakfast (B).

TABLE 5

| | NSC Sweetener | | | | Sucrose Control | | | |
|---|---|---|---|---|---|---|---|---|
| | 0 min | 120 min | D | B | 0 min | 120 min | D | B |
| Bloating | 5.8 | 10.87 | 14.43 | 11.79 | 7.93 | 4.67 | 5.43 | 5.71 |
| Belching | 0.7 | 0.80 | 4.14 | 2.43 | 0.60 | 0.40 | 1.79 | 0.57 |
| Nausea | 0.9 | 8.60 | 9.43 | 4.00 | 0.33 | 1.87 | 2.57 | 0.57 |
| Diarrhea | 0.7 | 3.53 | 19.21 | 8.93 | 0.27 | 0.80 | 6.93 | 5.79 |
| Flatulence | 7.6 | 4.60 | 14.79 | 7.36 | 1.47 | 1.40 | 8.93 | 7.50 |

Unlike the sucrose control, the results demonstrate that an NSC of the type disclosed herein did not raise postprandial glucose, insulin, or fructose levels after ingestion. There were no differences in palatability between the two test meals. The physical comfort scores tended to be higher after the ingestion of the NSC at most time point but only reaching statistical significance at 120 min for diarrhea.

What is claimed is:

1. A method of attenuating a postprandial insulin response comprising administering to a subject a composition comprising at least one sugar alcohol, stevia, monk fruit, a rheology modifier, and tagatose wherein a subject ingesting a carbohydrate-containing food comprising the composition experiences a decrease in the postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

2. The method of claim 1 wherein the subject ingesting a carbohydrate-containing food comprising the composition experiences a decrease in the postprandial insulin response of from about 20% to about 75% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

3. The method of claim 1 wherein the subject ingesting a carbohydrate-containing food comprising the composition experiences a decrease in a postprandial glucose response of from about 10% to about 90% for a time period ranging from about 15 minutes to about 180 minutes subsequent to ingestion of the carbohydrate-containing food when compared to the postprandial glucose response of a subject ingesting an otherwise identical carbohydrate-containing food comprising an equivalent amount of sucrose.

4. The method of claim 1 wherein the subject ingesting a carbohydrate-containing food comprising the composition experiences a decrease in a postprandial glucose response of from about 20% to about 75% for a time period ranging from about 15 minutes to about 180 minutes subsequent to ingestion of the carbohydrate-containing food when compared to the postprandial glucose response of a subject ingesting an otherwise identical carbohydrate-containing food comprising an equivalent amount of sucrose.

5. The method of claim 1 wherein a subject ingesting a carbohydrate-containing food comprising the composition experiences a decrease in a postprandial fructose response of from about 10% to about 90% for a time period ranging from about 15 minutes to about 180 minutes subsequent to ingestion of the carbohydrate-containing food when compared to the postprandial fructose response of a subject ingesting an otherwise identical carbohydrate-containing food comprising an equivalent amount of sucrose.

6. The method of claim 1 wherein a subject ingesting a carbohydrate-containing food comprising the composition experiences a decrease in a postprandial fructose response of from about 20% to about 75% for a time period ranging from about 15 minutes to about 180 minutes when compared to the postprandial fructose response of a subject ingesting an otherwise identical carbohydrate-containing food comprising an equivalent amount of sucrose.

7. The method of claim 1 wherein the subject is a human.

8. The method of claim 1 wherein the subject is diagnosed with diabetes mellitus type 2, obesity, hypertension, cardiovascular disease, lipidemia, hypertriacylglycerolemia, hypercholesterolemia, chronic kidney disease, or combinations thereof.

9. The method of claim 1 wherein the subject is administered from about 10 mg to about 1000 mg of the composition.

10. The method of claim 1 wherein the subject is administered from about 50 mg to about 500 mg of the composition.

11. The method of claim 1 wherein the composition is self-administered.

12. The method of claim 1 wherein the composition is administered at least once daily.

13. The method of claim 8 wherein the composition is administered at least once daily.

14. The method of claim 9 wherein the composition is administered at least once daily.

15. The method of claim 1 wherein the composition excludes allulose, xylose, inulin, chromium, valerian, or cellulose.

16. A method of attenuating a postprandial glucose response comprising administering to a subject a composition comprising at last one sugar alcohol, monk fruit, a rheology modifier, and tagatose wherein a subject ingesting (i) a carbohydrate-containing food and (ii) the composition experiences a decrease in a postprandial insulin response of from about 10% to about 90% for a time period ranging from about 15 minutes to about 180 minutes subsequent to ingestion of the composition when compared to the postprandial insulin response of a subject ingesting an otherwise identical carbohydrate-containing food comprising an equivalent amount of sucrose.

17. The method of claim 16 wherein the composition is provided as a dosage form selected from the group consisting of pills, buccal tablet, sub-lingual tablet, orally-disintegrating tablet, liquid, suspension, and powder.

18. The method of claim 16 wherein the composition excludes allulose, xylose, inulin, chromium, valerian, or cellulose.

19. The method of claim 16 wherein the subject ingests from about 10 mg to about 1000 mg of the composition.

20. The method of claim 16 wherein a subject experiences a decrease in a postprandial insulin response of from about 10% to about 90% for a time period ranging from about 15 minutes to about 180 minutes subsequent to ingestion of the carbohydrate-containing food when compared to the postprandial insulin response of a subject ingesting an otherwise identical carbohydrate-containing food comprising an equivalent amount of sucrose.

21. The method of claim 16 wherein a subject experiences a decrease in a postprandial fructose response of from about 10% to about 90% for a time period ranging from about 15 minutes to about 180 minutes subsequent to ingestion of the carbohydrate-containing food when compared to the postprandial fructose response of a subject ingesting an otherwise identical carbohydrate-containing food comprising an equivalent amount of sucrose.
